(19) United States
(12) Patent Application Publication
(10) Pub. No.: US 2025/0239062 A1
(43) Pub. Date: Jul. 24, 2025

(54) ULTRASOUND MACHINE LEARNING TECHNIQUES USING TRANSFORMED IMAGE DATA

(71) Applicant: GE Precision Healthcare LLC, Waukesha, WI (US)

(72) Inventor: Kyudong Kim, Seongnam-si (KR)

(21) Appl. No.: 18/420,193

(22) Filed: Jan. 23, 2024

(51) Int. Cl. G06V 10/82; G06T 7/00; G06V 10/774

(52) U.S. Cl. CPC G06V 10/82; G06T 7/0012; G06V 10/774; G06T 2207/20048; G06T 2207/20081; G06T 2207/20084; G06T 2207/30096; G06V 2201/031

(57) ABSTRACT

According to embodiments, a method for analyzing ultrasound image data obtained from ultrasonic imaging comprises: obtaining, by an ultrasound probe, the ultrasound image data; transforming, by a processor, the ultrasound image data with at least one transform to generate at least one set of transformed data; inputting the ultrasound image data and the at least one set of transformed data into a machine-learning model, wherein the machine-learning model is implemented by the processor; implementing, by the processor, the machine-learning model with the ultrasound image data and the at least one set of transformed data; and identifying, by the processor, at least one feature in the ultrasound image data as determined by the machine-learning model.

### FIELD

Certain embodiments relate to ultrasound imaging. More specifically, certain embodiments relate to techniques for determining one or more features in an ultrasound image by using transformed image data.

### BACKGROUND

Ultrasound imaging is a medical imaging technique for imaging human anatomy. Ultrasound imaging may be used to image or analyze blood flow through a patient's cardiovascular system. Ultrasound imaging uses real time, non-invasive high frequency sound waves to produce two-dimensional (2D), three-dimensional (3D), and/or four-dimensional (4D) (i.e., real-time/continuous 3D image data) image data.

Ultrasound systems obtain image data. For example, this image data may be B-mode data, which may show intensity of reflections at different spatial locations in a patient (hereinafter, spatial B-mode image or image data). This data may be used by a machine learning model to identify features in the ultrasound image. Such features include a patient's organs, including organs with lesions. Examples of such organs include liver, kidney, pancreas, or spleen. Techniques that improve the ability of a machine learning model to identify features, such as a patient's organ with a malignant lesion, may be helpful.

Further limitations and disadvantages of conventional and traditional approaches will become apparent to one of skill in the art, through comparison of such systems with some aspects of the present disclosure as set forth in the remainder of the present application with reference to the drawings.

### SUMMARY

According to embodiments, a method for analyzing ultrasound image data obtained from ultrasonic imaging comprises: obtaining, by an ultrasound probe, the ultrasound image data; transforming, by a processor, the ultrasound image data with at least one transform to generate at least one set of transformed data; inputting the ultrasound image data and the at least one set of transformed data into a machine-learning model, wherein the machine-learning model is implemented by the processor; implementing, by the processor, the machine-learning model with the ultrasound image data and the at least one set of transformed data; and identifying, by the processor, at least one feature in the ultrasound image data as determined by the machine-learning model. The method may further comprise determining an extent of the ultrasound image data according to a region of interest. The machine-learning model may include a convolutional neural network. The method may further comprise training the machine-learning model by: inputting annotated ultrasound image data into the machine-learning model, wherein the ultrasound image data indicates the presence or absence of the at least one feature; transforming the ultrasound image data using at least one transform to generate transformed data; and inputting the transformed data into the machine-learning model. The method may further comprise updating the machine-learning model to reduce a loss function as the machine-learning model receives additional ultrasound image data indicating the presence or absence of the at least one feature. The ultrasound image data may include spatial B-mode image data. The at least one set of transformed data may include at least one of Fourier transformed data, slant transformed data, or Hadamard transformed data. The at least one set of transformed data may include only one of Fourier transformed data, slant transformed data, or Hadamard transformed data. The at least one set of transformed data may include only two of Fourier transformed data, slant transformed data, or Hadamard transformed data. The at least one set of transformed data may include Fourier transformed data, slant transformed data, and Hadamard transformed data. The at least one feature may include at least one of an organ of a patient having no lesion or having a lesion (benign or malignant).

According to embodiments, a system for analyzing ultrasound image data obtained from ultrasonic imaging includes: an ultrasound probe configured to obtain the ultrasound image data; and a processor configured to transform the ultrasound image data with at least one transform to generate at least one set of transformed data, input the ultrasound image data and the at least one set of transformed data into a machine-learning model, wherein the machine-learning model is implemented by the processor, implement the machine-learning model with the ultrasound image data and the at least one set of transformed data, and identify at least one feature in the ultrasound image data as determined by the machine-learning model. The processor may be further configured to determine an extent of the ultrasound image data according to a region of interest. The machine-learning model may include a convolutional neural network. The processor may be configured to train the machine-learning model by inputting annotated ultrasound image data into the machine-learning model, wherein the ultrasound image data indicates the presence or absence of the at least one feature, transforming the ultrasound image data using at least one transform to generate transformed data, and inputting the transformed data into the machine-learning model. The processor may be configured to implement the machine-learning model to reduce a loss function as the machine-learning model receives additional ultrasound image data indicating the presence or absence of the at least one feature. The ultrasound image data may include spatial B-mode image data. The at least one set of transformed data may include at least one of Fourier transformed data, slant transformed data, or Hadamard transformed data. The at least one set of transformed data may include only one of Fourier transformed data, slant transformed data, or Hadamard transformed data. The at least one feature may include at least one of an organ of a patient being normal or having a lesion (e.g., benign or malignant).

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable for identifying features in ultrasound image data using a machine learning model, in accordance with various embodiments.

FIG. 2 is an exemplary spatial B-mode image and a region of interest (or ROI) therein.

FIG. 3A shows spatial B-mode image data in a region of interest.

FIGS. 3B-3D show transformations of spatial B-mode image data into different domains, in accordance with various embodiments.

FIG. 4 shows a block diagram of training or using a machine learning model to that processes spatial B-mode image data and at least one other transformed data, in accordance with various embodiments.

FIG. 5 is a flow chart illustrating exemplary steps that may be utilized for training a machine learning model to identify one or more types of features in spatial B-mode images, in accordance with various embodiments.

FIG. 6 is a flow chart illustrating exemplary steps that may be utilized for using a machine learning model to identify one or more types of features in spatial B-mode images, in accordance with various embodiments.

FIG. 7 shows a representation of a machine learning model to identify one or more types of features in spatial B-mode images, in accordance with various embodiments.

### DETAILED DESCRIPTION

Certain embodiments may be found in a method and system for identifying one or more features in spatial B-mode image data or other image data obtained by an ultrasound system. Such identification of feature(s) uses a trained machine learning model. Such a machine learning model (or more simply, model) may receive spatial B-mode image data and one or more transformed data as inputs. When training the model, the same type of data may be inputted to the model-spatial B-mode image data and one or more transformed data.

Aspects of the present disclosure have the technical effect of enhancing identification of features in ultrasound images using machine learning in order to help provide a diagnosis. Various embodiments have the technical effect of processing acquired ultrasound image data to identify features using machine learning. Certain embodiments have the technical effect of determining the probability of the existence of certain feature(s) in the ultrasound image data. Aspects of the present disclosure have the technical effect of improving identification of one or more features in ultrasound images. Aspects of the present disclosure have the technical effect of using transformed data to identify feature(s) in ultrasound images.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical, and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image (image data). However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising Brightness mode (B-mode or, also referred to as spatial B-mode), Motion mode (M-mode), Color Motion mode (CM-mode), Color Flow mode (CF-mode), Pulsed Wave (PW) Doppler, Continuous Wave (CW) Doppler, Contrast Enhanced Ultrasound (CEUS), and/or sub-modes of B-mode and/or CF-mode such as Harmonic Imaging, Shear Wave Elasticity Imaging (SWEI), Strain Elastography, Tissue Velocity Imaging (TVI), Power Doppler Imaging (PDI), B-flow, Micro Vascular Imaging (MVI), Ultrasound-Guided Attenuation Parameter (UGAP), and the like.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), Digital Signal Processor (DSP), Field-Programmable Gate Array (FPGA), Application-Specific Integrated Circuit (ASIC), or a combination thereof. A processor or processing unit may include multiple processors in the same location (e.g., integrated together in a single ASIC) or distributed over different locations. When there are multiple processors, they may communicate with other associated processors and/or work together to effect processing and computation.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

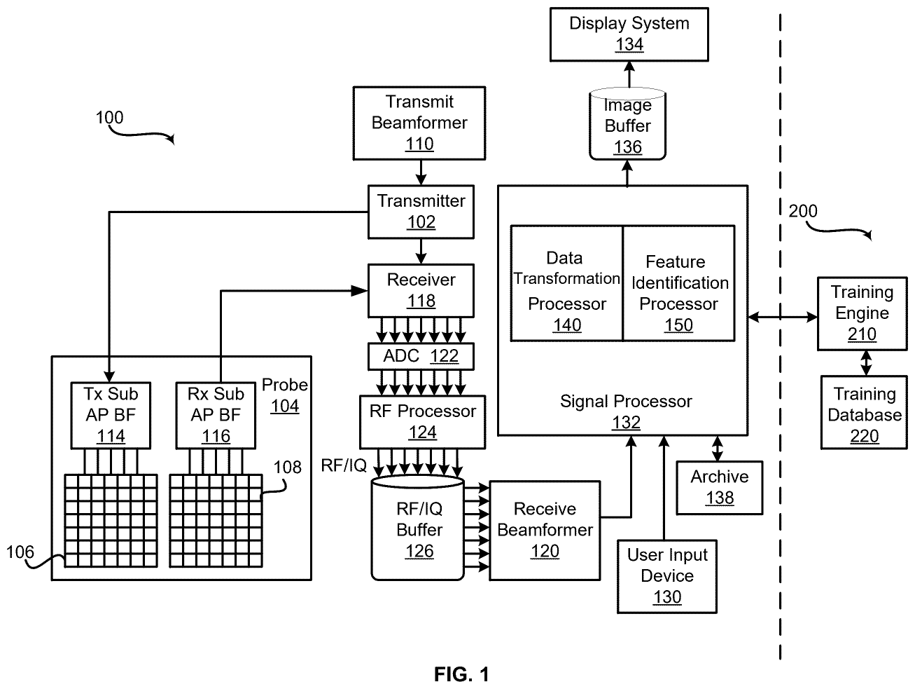

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system that is operable to identify features in image data obtained from a patient, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, analog-to-digital (A/D) converters 122, a radio frequency (RF) processor 124, a RF quadrature (RF/IQ) buffer 126, a user input device 130, a signal processor 132, an image buffer 136, a display system 134, and an archive 138.

The transmitter 102 may comprise suitable logic, circuitry, interfaces, and/or code that may be operable to drive an ultrasound probe 104. The ultrasound probe 104 may be a linear, convex, intracavitary, or phased array transducer. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. The group of transmit transducer elements 106 may emit ultrasonic signals through oil and a probe cap and into a target. In a representative embodiment, the ultrasound probe 104 may be operable to acquire ultrasound image data covering at least a substantial portion of an anatomy, such as a liver, kidney, pancreas, spleen, kidney, or any suitable anatomical structure. In an exemplary embodiment, the ultrasound probe 104 may be operated in a volume acquisition mode, where the transducer assembly of the ultrasound probe 104 acquires a plurality of parallel 2D ultrasound slices forming an ultrasound volume.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, and interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces, and/or code that may be operable to demodulate the digital signals output by the plurality of A/D converters 122. In accordance with an embodiment, the RF processor 124 may comprise a complex demodulator (not shown) that is operable to demodulate the digital signals to form me/Q data pairs that are representative of the corresponding echo signals. The RF or I/Q signal data may then be communicated to an RF/IQ buffer 126. The RF/IQ buffer 126 may comprise suitable logic, circuitry, interfaces, and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, select target structures for acquisition of images, input and/or select a region of interest, modify a region of interest, select regions of interest used to acquire images, a focused/zoomed volume, and the like. In an exemplary embodiment, the user input device 130 may be operable to configure, manage, and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera, and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (e.g., summed IQ signal) for generating ultrasound images for presentation on a display system 134. The signal processor 132 is operable to perform one or more processing operations according to a plurality of ultrasound modalities (such as B-mode, Doppler, and color Doppler modalities) on the acquired ultrasound scan data. In an exemplary embodiment, the signal processor 132 may be operable to perform display processing and/or control processing, among other things. Acquired ultrasound scan data, such as spatial B-mode data, may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise a data transformation processor 140 (or image data transformation processor 140), and a feature identification processor 150. The signal processor 132 may be capable of receiving input information from a user input device 130 and/or archive 138, generating an output displayable by a display system 134, and manipulating the output in response to input information from a user input device 130, among other things. The signal processor 132, the data transformation processor 140, and/or the feature identification processor 150 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 per second but may be lower or higher. As used herein, a “time” or “period of time” may correspond to one or more frames. The acquired ultrasound scan data may be displayed on the display system 134 at a display-rate that can be the same as the frame rate, or slower or faster. A sequence of images (for example of a patient's blood flow) may be displayed simultaneously. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a data transformation 140 that comprises suitable logic, circuitry, interfaces, and/or code that may be operable to use an ultrasound probe 104 to transform ultrasound image data. In an exemplary embodiment, the data transformation processor 140 may be configured to receive image data (e.g., spatial B-mode image data, or a portion thereof, such as data in a region of interest) and transform the image data using one or more transforms (e.g., Fourier transform, slant transform, and/or Hadamard transform, or Walsh transform). The data transformation processor 140 may be configured to receive a user input selecting a region of interest prior to performing an ultrasound image acquisition and analyzing the ultrasound image data and/or volume of the ultrasound image acquisition to obtain a sequence of images over time. The data transformation processor 140 may transform received image data for every one of the images or selected ones of the images.

The display system 134 may be any device capable of communicating visual information to a user. For example, a display system 134 may include a liquid crystal display, a light emitting diode display, and/or any suitable display or displays. The display system 134 can be operable to present 2D ultrasound images, 2D sequential ultrasound images, biplane ultrasound images, biplane ultrasound slices extracted from 3D/4D volumes, rendered 3D/4D volumes, selectable target structures, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores 2D ultrasound images, 2D sequential ultrasound images, biplane ultrasound images, biplane ultrasound slices extracted from 3D/4D volumes, rendered 3D/4D volumes, instructions for acquiring ultrasound image data, instructions for producing sequential ultrasound images, instructions for generating sample sequential ultrasound images, instructions for classifying images as generated or real, instructions for providing feedback based on the classifying of images, instructions for determining that an objective function has been reached, instructions for generating an enhanced sequential ultrasound image, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 210 may comprise suitable logic, circuitry, interfaces and/or code that may be operable to train the neurons of the deep neural network(s) (e.g., artificial intelligence model(s)) inferenced (i.e., deployed) by the data transformation processor 140, and/or the feature identification processor 150. For example, the machine-learning model implemented by feature identification processor 150 may be trained to identify features in spatial image data obtained by ultrasound system 100.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms. In some examples, the training image databases may include reference sequential ultrasound images of anatomical structures and/or tissues. In some examples, the reference sequential ultrasound images may be generated by the sequential image acquisition processor 140 and provided to the training image databases 220.

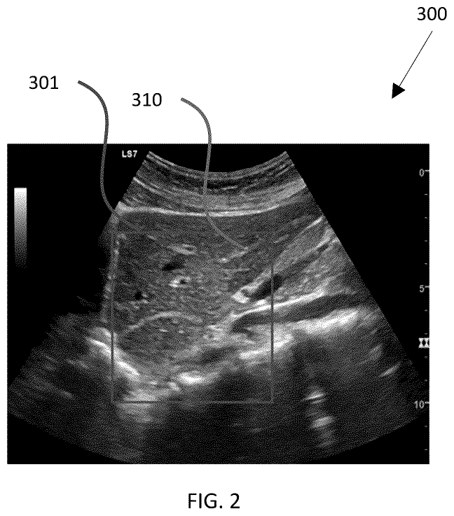

FIG. 2 is exemplary spatial B-mode image data 300 and a region of interest 301 therein. The spatial B-mode image data 300 shows an image including a region of a liver. The spatial B-mode image data 300 may be presented on a display system 134 for viewing by a user. The region of interest 301 defines a subset 310 of the spatial B-mode image data 300, and may be drawn and/or positioned by a user in the ultrasound image data 300 according to clinical purposes. The region of interest 301 may be drawn and/or positioned by the user through user input device 130. As generally disclosed herein, spatial B-mode image data 300 is used as an example, although other types of image data could be used in accordance with techniques described herein. Such other types of image data include Doppler image data or color Doppler image data. In systems that are multimodal (e.g., are capable of obtaining spatial B-mode image data and Doppler image data), multiple types of image data may be used, in addition to transformed data discussed further below.

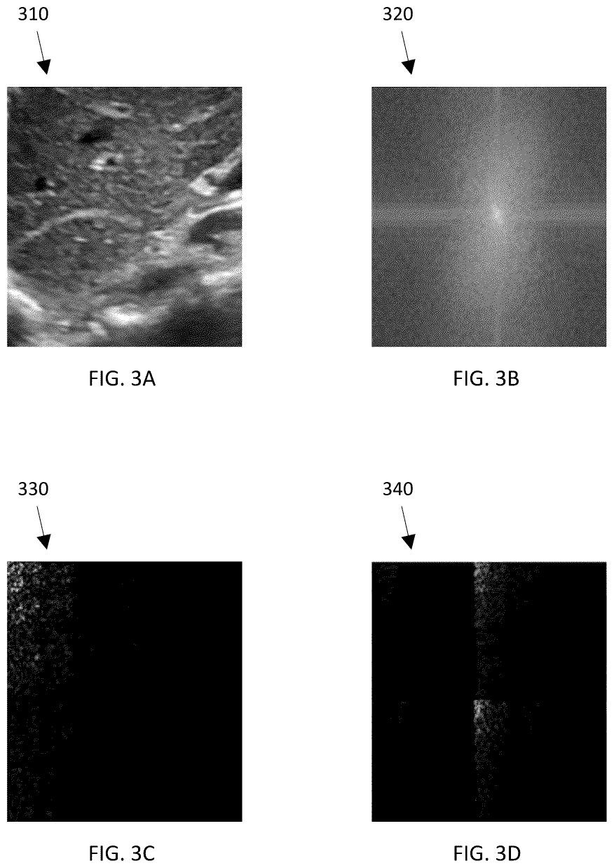

Referring again to FIG. 1, the data transformation processor 140 may be configured to gather ultrasound image data as the ultrasound probe 104 is glided across a region of interest (e.g., region of interest 310), an anatomical structure, tissues, and/or fluids contained therein (such as blood flowing through a region of interest of a patient's cardiovascular system). As the ultrasound probe 104 is glided across such a region, the data transformation processor 140 gathers ultrasound image(s) and transforms the image data according to one or more techniques. The data provided to the data transform processor 140 may be stored at archive 138 and/or any suitable computer readable medium, and the data transform processor 140 may obtain the ultrasound image data from the archive 138 and/or any suitable computer readable medium. Data transformation processor 140 may generate the images shown in FIGS. 3B, 3C, and 3D. FIG. 3A shows the subset of spatial B-mode image data 310 from FIG. 2.

FIG. 3B shows Fourier transformed data 320 that is the result of transformation of the subset of spatial B-mode image data corresponding to the region of interest 310 using a Fourier transform, and particularly a discrete Fourier transform. The particular Fourier transform data 320 shown in FIG. 3B is a graphical representation of the transformation of the subset of spatial B-mode image data 310 using a Fourier transform. In this case, a Fourier transform is a representation of an image as a sum of complex exponentials of varying magnitudes, frequencies, and phases. A two-dimensional discrete Fourier transform can be described by the following equation:

$${F}_{x} ( {K}_{1} , {K}_{2} ) = {{\sum }_{{n}_{1} = 0}}^{{N}_{1} - 1} {{\sum }^{{N}_{2} - 1}}_{{n}_{2} = 0} {f}_{x} ( {n}_{1} , {n}_{2} ) {e}^{- i \frac{2 π}{{N}_{1}} {n}_{1} {K}_{1} - i \frac{2 π}{{N}_{2}} {n}_{2} {K}_{2}}$$

Where K₁ is N₁−1, K₂ is N₂−1, N₁ and N₂ are integers corresponding to the size of the input image.

The data transformation processor 140 may be capable of implementing one or more such Fourier transforms on one or more types of inputted image data. For example, the data transformation processor 140 may transform image data 310 using two Fourier transforms to generate two sets of Fourier transform data 320, and where each of the Fourier transformed data 320 may be used in the same manner as the single Fourier transform data 320 depicted in FIG. 3B and used as an example herein. As another example, multiple Fourier transformed data 320 may be generated by the data transformation processor 140 using multiple inputs, such as spatial B-mode image data and Doppler image data. Image data from other modalities (e.g., color Doppler image data) could be included as part of this technique. Further, multiple Fourier transforms may be used for any given set of image data received by the data transformation processor 140. For example, data transformation processor 140 may use two types of Fourier transforms on spatial B-mode image data and two types of Fourier transforms on Doppler image data, resulting in four sets of transformed data. Image data obtained using different modalities may still correspond to the same region of interest 310.

FIG. 3C shows slant transformed data 330 that is the result of transformation of the subset of spatial B-mode image data corresponding to the region of interest 310 using a slant transform. The particular slant transform data 330 shown in FIG. 3C is a graphical representation of the transformation of the subset of spatial B-mode image data 310 using a slant transform. A slant transform can be described as follows. The slant transform, as indicated by its name, possesses a “slant” or a stair-like waveform for its first sequency vector. The matrix of slant transform may be composed by multiplying a series of sparse matrices on the Hadamard matrix. This will be shown as follows. In the following example, let S*_{N} denote the slant matrix of order N. The asterisk is used to represent that the sequency order is the same as the Hadamard matrix of the same order N. The subscript denotes the order of the matrix. Where N=2, S*_{N} is given by:

$${S}_{2}^{*} = \frac{1}{\sqrt{2}} [ \begin{matrix}1 & 1 \\ 1 & - 1\end{matrix} ]$$

Where N=4, S*_{N} is given by:

$${S}_{4}^{*} = \frac{1}{{2}^{1 / 2}} [ \begin{matrix}1 & & & \\ & {a}_{4} & - {b}_{4} & \\ & {b}_{4} & {a}_{4} & \\ & & & 1\end{matrix} ] [ \begin{matrix}{S}_{2}^{*} & {S}_{2}^{*} \\ {S}_{2}^{*} & - {S}_{2}^{*}\end{matrix} ]$$

The data transformation processor 140 may be capable of implementing one or more such slant transforms on one or more types of inputted image data. For example, the data transformation processor 140 may transform image data 310 using two slant transforms to generate two sets of slant transformed data 330, and where each of the slant transformed data 330 may be used in the same manner as the single slant transformed data 330 depicted in FIG. 3C and used as an example herein. As another example, multiple slant transformed data 330 may be generated by the data transformation processor 140 using multiple inputs, such as spatial B-mode image data and Doppler image data. Image data from other modalities (e.g., color Doppler image data) could be included as part of this technique. Further, multiple slant transforms may be used for any given set of image data received by the data transformation processor 140. For example, data transformation processor 140 may use two types of slant transforms on spatial B-mode image data and two types of slant transforms on Doppler image data, resulting in four sets of transformed data. Image data obtained using different modalities may still correspond to the same region of interest 310.

FIG. 3D shows Hadamard transformed data 340 that is the result of transformation of the subset of spatial B-mode image data corresponding to the region of interest 310 using a Hadamard transform. The particular Hadamard transformed data 340 shown in FIG. 3D is a graphical representation of the transformation of the subset of spatial B-mode image data 310 using a Hadamard transform.

The Hadamard transform Hₘ is a 2^{m}×2^{m} matrix, that transforms 2^{m} real numbers xₙ into 2 real numbers elements Xₖ. The Hadamard transform can be defined in two ways: recursively, or by using the binary (base-2) representation of the indices n and k. Recursively, we define a 1×1 Hadamard transform H₀ by the identity H₀=1, and then define Hₘ for m>0 by:

$${H}_{m} = \frac{1}{\sqrt{2}} ( \begin{matrix}{H}_{m - 1} & {H}_{m - 1} \\ {H}_{m - 1} & - {H}_{m - 1}\end{matrix} )$$

where 1 over the square root of 2 is a normalization factor that may be omitted.

For M>1, Hₘ can also be defined by:

$${H}_{m} = {H}_{m} ⊗ {H}_{m - 1}$$

Where
⊗
represents the Kronecker product

Thus. other than normalization factor, the Hadamard matrices may be made up entirely of 1 and −1. Further, the Hadamard matrix may be defined by its (k, n)-th entry by:

$$k = {\sum }_{i = 0}^{m - 1} {k}_{i} {2}^{i} = {k}_{m - 1} {2}^{m - 1} + {k}_{m - 2} {2}^{m - 2} + ⋯ + {k}_{1} {2}_{+} {k}_{0}$$

$$n = {\sum }_{i = 0}^{m - 1} {n}_{i} {2}^{i} = {n}_{m - 1} {2}^{m - 1} + {n}_{m - 2} {2}^{m - 2} + ⋯ + {n}_{1} {2}_{+} {n}_{0}$$

Where the kⱼ and nⱼ are the bit elements (0 or 1) or k and n, respectively.

For the element in the top left corner, k=n=0. This results in:

$${( {H}_{m} )}_{k , n} = \frac{1}{{2}^{m / 2}} {( - 1 )}^{{\sum }_{j} {k}_{j} {n}_{j}}$$

The data transformation processor 140 may be capable of implementing one or more such Hadamard transforms on one or more types of inputted image data. For example, the data transformation processor 140 may transform image data 310 using two Hadamard transforms to generate two sets of Hadamard transformed data 340, and where each of the Hadamard transformed data 340 may be used in the same manner as the single Hadamard transformed data 340 depicted in FIG. 3D and used as an example herein. As another example, multiple Hadamard transformed data 340 may be generated by the data transformation processor 140 using multiple inputs, such as spatial B-mode image data and Doppler image data. Image data from other modalities (e.g., color Doppler image data) could be included as part of this technique. Further, multiple Hadamard transforms may be used for any given set of image data received by the data transformation processor 140. For example, data transformation processor 140 may use two types of Hadamard transforms on spatial B-mode image data and two types of Hadamard transforms on Doppler image data, resulting in four sets of transformed data. Image data obtained using different modalities may still correspond to the same region of interest 310.

While embodiments herein describe Fourier transforms, slant transforms, and Hadamard transforms, other types of transforms may be used as part of or in conjunction with the techniques described herein. Such transforms may also include a Hartley transform.

The feature identification processor 150 may comprise suitable logic, circuitry, interfaces, and/or code that may be operable to identify one or more features from the image data provided to data transformation processor 140 and the transformed data generated by the data transformation processor 140. The feature identification processor 150 may identify one or more features only from the transformed data provided by the data transformation processor 140, and not the original image data (e.g., spatial B-mode image data). The feature identification processor 150 may identify feature(s) from both the transformed data provided by the data transformation processor 140 and the original image data (e.g., spatial B-mode image data). Such feature(s) may be identified by using statistical values—e.g., a 20%, 40%, 60%, or 80% likelihood that a feature exists in the spatial B-mode image data. Multiple features may be identified, and the feature with the greatest likelihood may be selected. For example, if there is a 95% chance that the image data indicates a malignant lesion and a 5% chance that the image data indicates a benign lesion, then the malignant lesion will be identified.

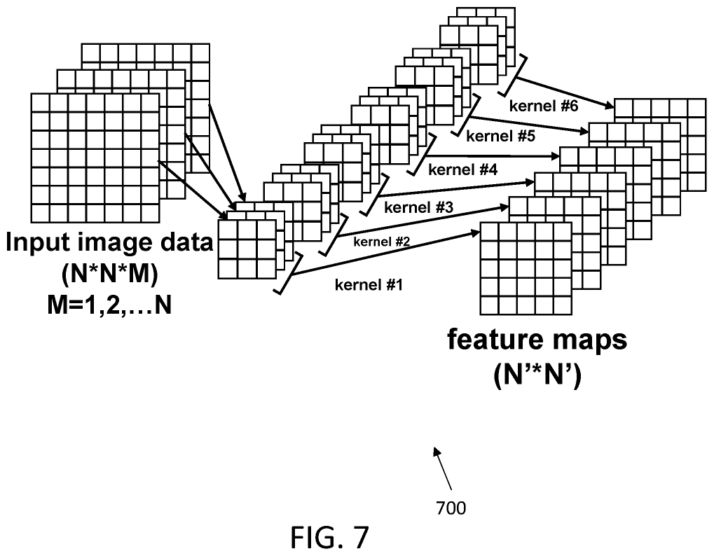

The feature identification processor 150 may implement a machine-learning model to identify features in the spatial B-mode image data. Such a model may include a convolutional neural network (or CNN). Such a convolutional neural network model may be three dimensional. FIG. 7 illustrates a machine-learning model 700, which is a convolutional neural network. The machine-learning model 700 receives M sets of input image data. The input image data may correspond to a limited amount of the underlying spatial image data acquired by the ultrasound system. For example, input image data may correspond to or be limited to a region of interest, such as region of interest 301 shown in FIG. 2.

The format of the input image data may be adjusted for the purpose of generating a useful feature map. As shown, multiple input image data may be received by the machine-learning model 700. The image data may include one or more transformed data sets (e.g., Fourier, slant, and/or Hadamard) and optionally in combination with the spatial image data (e.g., B-mode, Doppler, color Doppler). One or more different kernels or filters may be applied to the input image data. The kernels may be three-dimensional, and may have a depth equal to the depth of the input image data. As shown in FIG. 7, each of the input data and the kernels have a depth of three layers, although two dimensional kernels are possible too. The filtered input image data may result in a corresponding number of feature maps. The feature maps may be two-dimensional (as shown) or one-dimensional. The feature maps may further be processed or assessed to determine the likelihood of the existence of a given feature—e.g., an organ with a benign lesion or malignant lesion.

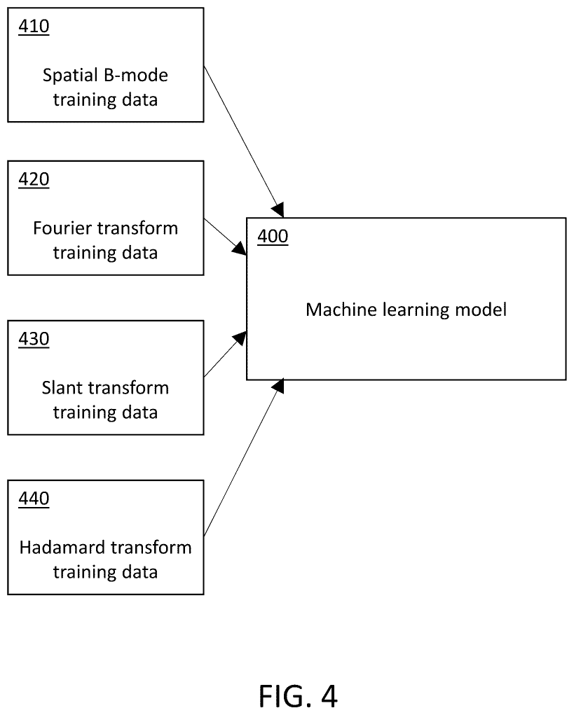

An example of training a machine-learning model 400 for use by feature identification processor 150 is shown in FIG. 4. The model 400 may be trained through a process of supervised learning, in which the training data (410, 420, 430, and/or 440) are labeled or annotated. Training may take place on the training system 200. The training data may be stored in training database 220, and processed by training engine 210. All of the training data may not be stored in the training database 220. The spatial image training data may be stored in the training database 220, and the transformed image training data may be determined from the stored spatial image training data by a processor such as data transformation processor 140.

The machine-learning model 400 may receive input image data, including spatial B-mode training data 410, Fourier transform training data 420, slant transform training data 430, and/or Hadamard transform training data 440. The machine-learning model 400 may be similar to machine-learning model 700. Learning may be performed in two steps. First, input image data 410, 420, 430, and/or 440 may be inputted into machine learning model 400, which then processes the input image data by a network of neurons to generate an output vector. The highest value of the output vector represents the detected object class, such as a kidney with a malignant lesion. This process may be referred to as feedforward. This detection may or may not be correct. A loss function may then be determined based on the target value of the output of the machine learning model 400 and the actual value that was outputted. The actual value is known because the training data is labeled or annotated. The loss function may include some or all elements and parameters of the neural network. These parameters are updated to minimize or reduce the loss function. This process may be referred to as backpropogation. The feedforward and backpropogation processes may be iterated one or more times until the machine-learning model 400 becomes sufficiently trained. The training may take place on the training system 200. The trained machine-learning model 400 may then be stored on ultrasound system 100.

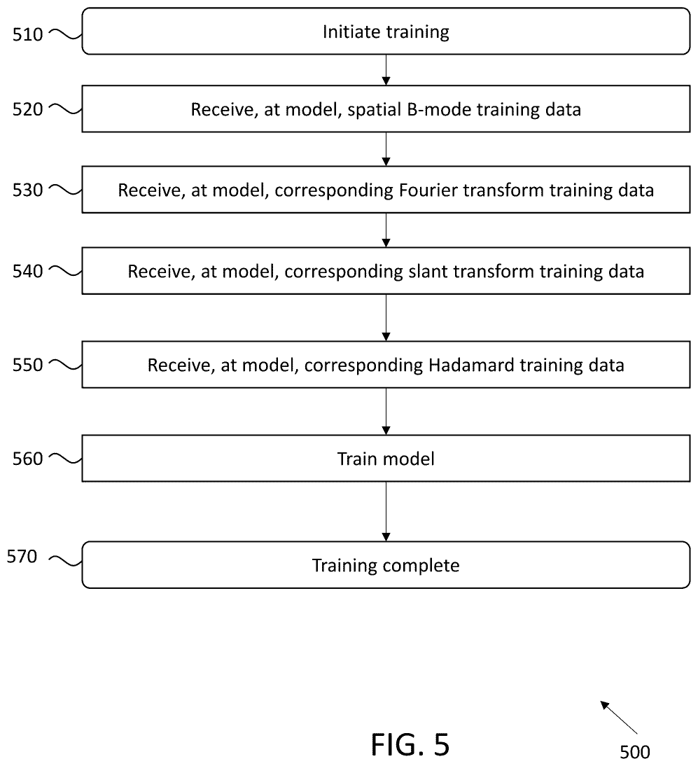

FIG. 5 is a flow chart 500 illustrating exemplary steps that may be utilized for training a machine learning model to identify one or more types of features in spatial B-mode images, in accordance with various embodiments. Examples of a machine-learning model are described in context of machine-learning models 400, 700. The steps in the flowchart 500 may be performed in a different order, or some steps may be omitted. The flowchart may be performed by training engine 210. The training data described in the flowchart 500 may be stored in training database 220. The machine-learning model may be or include a convolutional neural network.

At step 510, training of a machine-learning model is initiated by a processor, such as a processor in training engine 210. At step 520, spatial B-mode training data (such as data 410) is received at the machine-learning model. Exemplary spatial B-mode training data is described in conjunction with data 310, 410. Other spatial data, such as Doppler or color Doppler image data may be used instead of or in addition to the spatial B-mode training data. The data may be labeled or annotated. For example, each image may be labeled as a particular type of organ and whether the organ has or does not have an abnormality such as a benign lesion or malignant lesion. Annotations may be performed manually to generate the training data for the spatial B-mode training data.

At step 530, Fourier transform training data (such as data 420) is received at the machine-learning model. Exemplary Fourier transform training data is described in conjunction with data 320, 420. The Fourier transform training data may be labeled or annotated. The annotations or labels from the original spatial image data may be carried over to the Fourier transform training data. The Fourier transform training data may be stored in training database 220, or may be transformed from spatial image data stored in the training database 220 before being inputted into the machine-learning model. In other words, it may not be necessary to store the Fourier transform training data in training database 220.

At step 540, slant transform training data (such as data 430) is received at the machine-learning model. Exemplary slant transform training data is described in conjunction with data 330, 430. The slant transform training data may be labeled or annotated. The annotations or labels from the original spatial image data may be carried over to the slant transform training data. The slant transform training data may be stored in training database 220, or may be transformed from spatial image data stored in the training database 220 before being inputted into the machine-learning model. In other words, it may not be necessary to store the slant transform training data in training database 220.

At step 550, Hadamard transform training data (such as data 440) is received at the machine-learning model. Exemplary Hadamard transform training data is described in conjunction with data 340, 440. The Hadamard transform training data may be labeled or annotated. The annotations or labels from the original spatial image data may be carried over to the Hadamard transform training data. The Hadamard transform training data may be stored in training database 220, or may be transformed from spatial image data stored in the training database 220 before being inputted into the machine-learning model. In other words, it may not be necessary to store the Hadamard transform training data in training database 220.

At step 560, the machine-learning model is trained. The machine-learning model may be trained in accordance with the techniques described in conjunction with FIGS. 4 and 7. Training continues until step 570, at which time training is complete. The trained machine-learning model may be stored in ultrasound system 100 and may be implemented by feature identification processor 150.

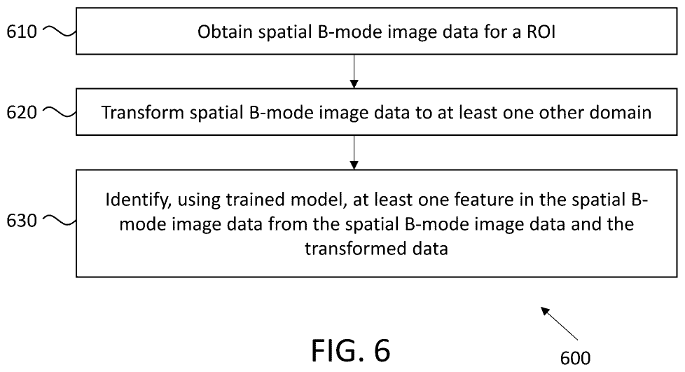

FIG. 6 is a flowchart 600 illustrating exemplary steps that may be utilized for using a machine-learning model (e.g., machine-learning models 400, 700) to identify one or more types of features in spatial B-mode images, in accordance with various embodiments. The machine-learning model has already been trained. The flowchart 600 may be implemented by ultrasound system 100, including data transformation processor 140 and feature identification processor 150.

At step 610, spatial B-mode image data for a region of interest may be obtained. Techniques for obtaining spatial B-mode image data as well as determining a region of interest therein are described above in conjunction with ultrasound system 100. An exemplary region of interest 310 of spatial B-mode image data 300 is described above in context of FIGS. 2 and 3A

At step 620, the spatial B-mode image data may be transformed into at least one other domain (e.g., Fourier, slant Hadamard). Transformation may be performed by data transformation processor 140, as described above.

At step 630, at least one feature in the spatial B-mode image data may be identified using the trained model. The feature(s) may be identified using feature identification processor 150. The transformed data and optionally the spatial B-mode image data for the region of interest may be received by the trained machine-learning model (such as machine-learning models 400, 700). The trained machine-learning model may output a likelihood of a given feature—e.g., an 80% chance that there is a malignant lesion on a patient's kidney, a 19% chance that there is a benign lesion, and a 1% chance that there is no lesion.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for enhancing sequential ultrasound images using deep learning.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

1. A method for analyzing ultrasound image data obtained from ultrasonic imaging, the method comprising:
obtaining, by an ultrasound probe, the ultrasound image data;
transforming, by a processor, the ultrasound image data with at least one transform to generate at least one set of transformed data;
inputting the ultrasound image data and the at least one set of transformed data into a machine-learning model, wherein the machine-learning model is implemented by the processor;
implementing, by the processor, the machine-learning model with the ultrasound image data and the at least one set of transformed data; and
identifying, by the processor, at least one feature in the ultrasound image data as determined by the machine-learning model.

2. The method of claim 1, further comprising determining an extent of the ultrasound image data according to a region of interest.

3. The method of claim 1, wherein the machine-learning model comprises a convolutional neural network.

4. The method of claim 3, further comprising training the machine-learning model by:
inputting annotated ultrasound image data into the machine-learning model, wherein the ultrasound image data indicates the presence or absence of the at least one feature;
transforming the ultrasound image data using at least one transform to generate transformed data; and
inputting the transformed data into the machine-learning model.

5. The method of claim 4, further comprising updating the machine-learning model to reduce a loss function as the machine-learning model receives additional ultrasound image data indicating the presence or absence of the at least one feature.

6. The method of claim 1, wherein the ultrasound image data comprises spatial B-mode image data.

7. The method of claim 1, wherein the at least one set of transformed data comprises at least one of Fourier transformed data, slant transformed data, or Hadamard transformed data.

8. The method of claim 7, wherein the at least one set of transformed data comprises only one of Fourier transformed data, slant transformed data, or Hadamard transformed data.

9. The method of claim 7, wherein the at least one set of transformed data comprises only two of Fourier transformed data, slant transformed data, or Hadamard transformed data.

10. The method of claim 7, wherein the at least one set of transformed data comprises Fourier transformed data, slant transformed data, and Hadamard transformed data.

11. The method of claim 1, wherein the at least one feature comprises at least one of an organ of a patient having a benign lesion or a malignant lesion.

12. A system for analyzing ultrasound image data obtained from ultrasonic imaging, the system comprising:
an ultrasound probe configured to obtain the ultrasound image data; and
a processor configured to transform the ultrasound image data with at least one transform to generate at least one set of transformed data, input the ultrasound image data and the at least one set of transformed data into a machine-learning model, wherein the machine-learning model is implemented by the processor, implement the machine-learning model with the ultrasound image data and the at least one set of transformed data, and identify at least one feature in the ultrasound image data as determined by the machine-learning model.

13. The system of claim 12, wherein the processor is further configured to determine an extent of the ultrasound image data according to a region of interest.

14. The system of claim 12, wherein the machine-learning model comprises a convolutional neural network.

15. The method of claim 14, wherein the processor is further configured to train the machine-learning model by inputting annotated ultrasound image data into the machine-learning model, wherein the ultrasound image data indicates the presence or absence of the at least one feature, transforming the ultrasound image data using at least one transform to generate transformed data, and inputting the transformed data into the machine-learning model.

16. The method of claim 15, wherein the processor is further configured to implement the machine-learning model to reduce a loss function as the machine-learning model receives additional ultrasound image data indicating the presence or absence of the at least one feature.

17. The system of claim 12, wherein the ultrasound image data comprises spatial B-mode image data.

18. The system of claim 12, wherein the at least one set of transformed data comprises at least one of Fourier transformed data, slant transformed data, or Hadamard transformed data.

19. The method of claim 12, wherein the at least one set of transformed data comprises only one of Fourier transformed data, slant transformed data, or Hadamard transformed data.

20. The method of claim 1, wherein the at least one feature comprises at least one of an organ of a patient having a benign lesion or a malignant lesion.
